(19) United States
(12) Patent Application Publication
(10) Pub. No.: US 2013/0023757 A1
(43) Pub. Date: Jan. 24, 2013

(54) INTERCONNECT FOR AN MRI DETECTOR COIL MOUNTED ON A CATHETER

(71) Applicants: Richard Rodney Anthony Syms, Ealing (GB); Ian Robert Young, Marlborough (GB); Muhammad Munir Ahmad, Egham (GB)

(72) Inventors: Richard Rodney Anthony Syms, Ealing (GB); Ian Robert Young, Marlborough (GB); Muhammad Munir Ahmad, Egham (GB)

(73) Assignee: IMPERIAL INNOVATIONS LIMITED, London (GB)

(21) Appl. No.: 13/574,107

(22) PCT Filed: Jan. 18, 2011
(86) PCT No.: PCT/GB2011/050075

§ 371 Date: Oct. 11, 2012

(30) Foreign Application Priority Data
Jan. 19, 2010 (GB) 1000813.4

(51) Int. Cl. H01P 5/08; A61B 5/055; G01R 33/341

(52) U.S. Cl. 600/423; 333/260; 324/322

(57) ABSTRACT

An interconnect for an RF detector coil comprises a substrate comprising strip of insulating material, a conducting layer formed on a first side of the substrate, and a ground layer formed on a second side of the substrate. At least one of the layers has a periodic pattern so that the conducting layer alternates along its length between capacitive regions which are opposite regions of the ground layer, and non-capacitive regions.

### FIELD OF THE INVENTION

The present invention relates to detectors, and has particular application in RF detectors for example for magnetic resonance imaging (MRI) scanners.

### BACKGROUND TO THE INVENTION

Many small resonant radio-frequency (RF) detectors have been developed for internal imaging in in-vivo magnetic resonance imaging (MRI), to take advantage of the improved signal-to-noise ratio offered by close proximity to the signal source. Similar devices have been used for catheter tracking. Common features of such coils are hand-made construction, based on wire-wound coils and discrete capacitors. For applications such as intravascular imaging, the coils are typically mounted on a catheter, with a sub-miniature co-axial output being taken through an internal lumen. Individual tuning and matching is required, and the assemblies are fragile, bulky and lack the reproducibility needed for large-scale clinical use.

In recent years, increasing use has been made of micro-fabricated coils, formed by electroplating of Cu on flexible substrates such as polyimide and PTFE. These coils are more reproducible, and image quality is high but assembly still requires discrete capacitors. Coils with integrated capacitors are now being developed, and we have recently demonstrated a method of tuning and matching that allows a complete RF resonator to be formed using double-sided patterning of copper-clad polyimide [GB 0910039.7]. The application was an endoscopically delivered, catheter-based detector for in-vivo imaging of the bile duct. However, the sub-miniature coaxial cable used to transmit the detected signal back along the catheter blocks one of the internal lumens of the catheter, which in a clinical setting are typically required for use with a guide-wire or for injection of contrast agent. In addition, the presence of a soldered joint involves comprehensive sealing for use in a wet environment.

### SUMMARY OF THE INVENTION

The present invention provides an interconnect for a RF detector coil. The interconnect comprises a substrate, which may comprise a strip of insulating material, a conducting layer formed on a first side of the substrate, and a ground layer, which may be formed on a second side of the substrate. At least one of the layers may have a periodic pattern. The conducting layer may alternate along its length between capacitive regions, which may be opposite regions of the ground layer, and non-capacitive regions.

The second side of the substrate may be only part covered by the ground layer, and in the non-capacitive regions the conducting layer may be opposite parts of the second side which are not covered by the ground layer. For example the ground layer may have openings in it.

The substrate may be flexible, which can allow the interconnect to be wrapped around an implement, which may be a surgical implement such as a catheter.

The conducting layer may be in the form of a straight strip. The ground layer may be periodically patterned so as to provide the capacitive and non-capacitive regions of the conducting layer. Alternatively the conducting layer may be patterned, and the ground layer may be either periodically patterned or straight.

The ground layer may comprise two spaced apart longitudinal strips extending along the substrate with cross strips extending across the substrate between the longitudinal strips.

The present invention further provides an RF detector coil assembly comprising an interconnect according to the invention and a detector coil. The detector coil may be connected to the interconnect. The coil may have two ends, one of which may be connected to the conducting layer and the other of which may be connected to the ground layer. The coil may be formed on a flexible substrate. For example the coil may be formed on the strip of insulating material which forms the substrate of the interconnect.

The present invention further provides a catheter assembly comprising a catheter having a cylindrical body with an interconnect according to the invention wrapped around its outer surface. The catheter assembly may further comprise a detector coil connected to the interconnect, which may be wrapped around its outer surface.

The catheter assembly may further comprise a protective sleeve enclosing the interconnect and the catheter body, and also the detector coil.

The entire structure may then be fabricated as a continuous structure and wrapped around the outside of a catheter, avoiding the need for an internal coaxial cable and simplifying sealing.

Geometric constraints make it difficult to achieve the required characteristic impedance using either a microstrip or a coplanar waveguide interconnect. Some embodiments of the invention achieve impedance matching by periodically patterning the ground plane of a microstrip. This approach has previously been used to form photonic bandgap filters but we have found that it can also be used to modify impedance at low frequency.

Some embodiments of the invention can provide lengths of the order of two metres of flexible interconnect with an overall thickness less than 100 μm, combined with thin film RF resonators to form catheter mounted flexible detector systems for ¹H MR imaging at 1.5 T.

Preferred embodiments of the present invention will now be described by way of example only with reference to the accompanying drawings.

### BRIEF DESCRIPTION OF THE DRAWINGS

FIG. 1 is a top view of a thin film interconnect in the form of a microstrip;

FIG. 1a is a section through the interconnect of FIG. 1;

FIG. 2 is a top view of a thin film interconnect in the form of a coplanar waveguide;

FIG. 2a is a section through the interconnect of FIG. 2;

FIG. 3 is a top view of a thin film interconnect with periodically patterned ground plane according to an embodiment of the invention;

FIG. 3a is a section through the interconnect of FIG. 3;

FIG. 4 is a top view of a thin film meander-line interconnect according to a further embodiment of the invention;

FIG. 5 is a top view of a thin-film resonant detector system according to an embodiment of the invention;

FIGS. 5a and 5b are sections through the system of FIG. 5 integrated onto a catheter;

FIG. 6 is a graph showing variation of impedance with strip width w in the interconnect of FIG. 1 for metal thickness t=35μm and different values of substrate thickness h;

FIG. 7 is a graph showing variation of impedance with strip width w in the interconnect of FIG. 2 for different values of ground-plane separation y;

FIG. 8 is a dispersion diagram for a bi-periodic L-C ladder network such as that of FIG. 3;

FIG. 9 is a graph showing frequency variation of the scattering parameter S₁₁, for an L-C ladder with different values of the impedance ratio Z′/Z_{L};

FIG. 10 is a photograph of experimental prototypes of thin-film interconnects similar to that of FIG. 3;

FIG. 11 is a photograph of experimental prototypes of thin film RF resonators similar to that of FIG. 5;

FIG. 12 is a graph showing an experimentally measured frequency variation of S₁₁ for uniform microstrip and for microstrip with a periodically patterned ground plane;

FIG. 13 is a graph showing an experimentally measured frequency variation of S₂₁ for uniform microstrip and for microstrip with a periodically patterned ground plane;

FIG. 14 is a graph showing frequency variation of S₁₁ and S₂₁ (assuming inductive excitation) for a complete catheter-based thin-film resonant RF microcoil detector system according to the invention; and

FIG. 15 is a 1H MR image of a duct in a lamb's liver taken using a detector coil and interconnect according to an embodiment of the invention.

### DESCRIPTION OF THE PREFERRED EMBODIMENTS

Referring to FIGS. 1 to 4, different interconnect formats will first be described. FIGS. 1 and 1a show an interconnect 10 having a microstrip geometry and comprising two conducting layers 12, 14 separated by an insulator layer 16 of relative dielectric constant εᵣ. One of the conducting layers 12 is patterned into a strip of width w extending along the centre of one side of the insulator strip 16, and the other 14 covers the whole of the other side of the insulator strip 16 forming a ground layer. The thicknesses of conductor layers and the insulator strip are t and h respectively. The characteristic impedance Z₀ of such a line can be estimated using analytic approximations developed for numerical values (see Wheeler H. A. “Transmission-line properties of a strip on a dielectric sheet on a plane” IEEE Trans. Micr. Theory & Tech. MTT-25, 631-647 (1977)) namely:

Z₀=Z_{FS} c₁ logₑ{1+c₂[c₂c₃+√(c₂²c₃²+c₄)]} (1)

Here Z_{FS}=√(μ₀/ε₀) is the impedance of free space and:

c₁=1/{2π√[2(1+εᵣ)]}

c₂=4h/w_{eff}

c₃=(14+8/εᵣ)/11

c₄=π²(1+1/εᵣ)/2

w_{eff}=w+t×c₅ logₑ{4e/√(c₆²+c₇²)}

c₅=(1+1/εᵣ)2π

c₆=t/h

c₇=1/{π(w/t+11/10)}² (2)

These expressions give the typical results shown in FIG. 6. Here it has been assumed that t=35 μm and εᵣ=3.5, to model double-sided flexible PCB based on a Cu-polyimide-Cu trilayer. Two curves are shown, for h =25 μm and h=50 μm, and 50 Ω impedance is only obtained for very small (<100 μm) strip widths, with w reducing as h decreases. The explanation is the small separation of the conductors in a flexible substrate, which results in a low inductance and a high capacitance per unit length. Lithographic fabrication of micro-strip that can be matched to a typical system impedance in the approximately 2 metre lengths needed for a catheter is therefore likely to be difficult. Larger inductance and/or smaller capacitance are required, but using geometries involving moderate (w≈1 mm) strip widths.

Referring to FIG. 2 a second design of interconnect 20 has a coplanar waveguide (CPW) geometry consisting of a central strip 22, of width w, extending along the centre of one side of an insulating strip 26, with conducting ground strips 24 on either side of, and spaced from, the central strip 22, and on the same side of the backing strip 26 to form a ground-signal-ground arrangement. The ground strips are spaced from each other by a gap of width y, within which the central strip is located. In this case, Z₀ of the interconnect 20 is found by first evaluating the capacitance Cₚ per unit length by conformal mapping, and then assuming that the effective dielectric constant is the average of that of the substrate 26 and any overlayer. Assuming that the latter is air, the result (see Wen C. P. “Coplanar waveguide: a surface strip transmission line suitable for nonreciprocal gyromagnetic device applications” IEEE Trans. Microwave Theory and Tech. MTT-17, 1087-1990 (1969)) is:

Z₀=1/Cₚvₚₕ,

where vₚₕ={(2/(εᵣ+1)}^{1/2}c ,

C=(εᵣ+1)ε₀ 2r (3)

Here c is the velocity of light, r=K(k)/K′(k) and K(k) is a complete elliptic integral of the first kind with modulus k such that:

K(k)=₀∫^{π/2}{1−k² sin²(θ)}^{-1/2} dθ,

k=w/y, K′(k)=K(k′), k′=(1−k²)^{1/2} (4)

FIG. 7 shows the variation of impedance with w for different values of the ground-plane separation y, again assuming that εᵣ=3.5. Although larger impedance may now be obtained using wider strips, 50Ω impedance is only obtained when w≈0.85 y. The explanation is the much smaller inter-electrode capacitance and larger inductance obtained in a CPW. Consequently, this geometry requires small electrode gaps (y−w)/2 which are again hard to manufacture. Furthermore, C_{P} is now sensitive to the dielectric constant of the overlayer, which may vary significantly if interconnects without a fixed cladding are immersed in tissue.

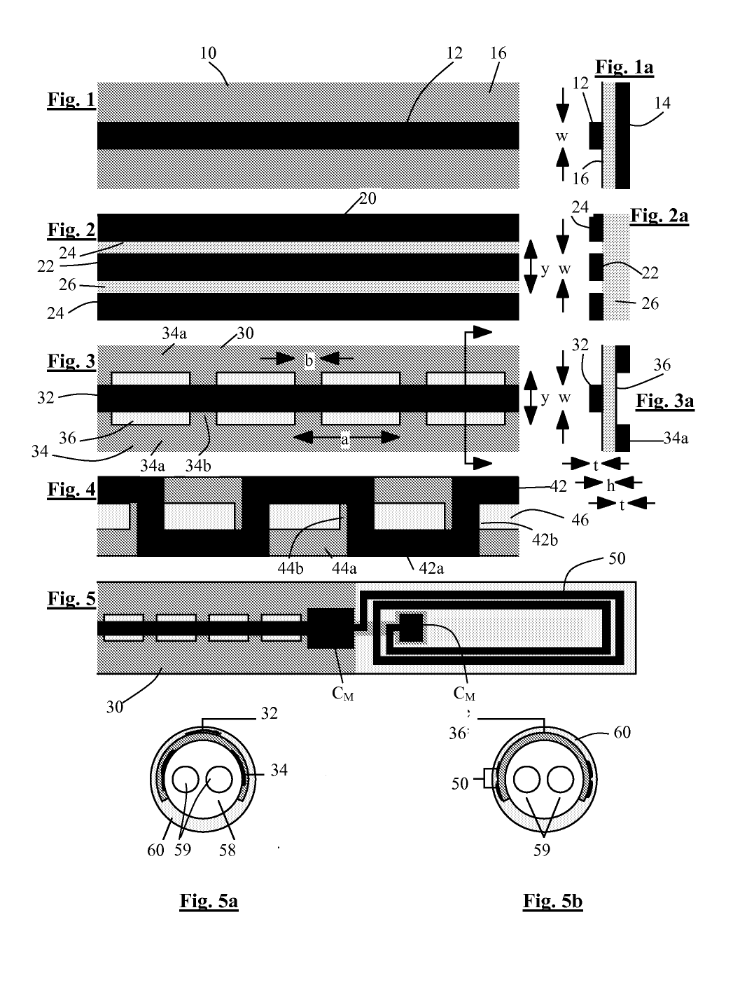

Referring to FIGS. 3 and 3a, a third design of interconnect 30 according to an embodiment of the invention combines some of the properties of micro-strip and CPW. In this embodiment a central conducting strip 32 of width w and thickness t is again provided on one side of an insulating strip 36, with a ground layer 34 of conducting material on the other side of the insulating strip 34. The ground layer 34 comprises two side strips 34a extending down the two sides of the insulating strip and a series of cross strips 34b extending across the insulating strip 36 between the two side strips 34a, and connected to the sides strips at each end so as to form a ladder structure. The width y of the spaces between the pairs of adjacent cross strips 34b, i.e. the distance by which the side strips 34b are spaced from each other, is greater than the width w of the central strip 32. The central strip 32 and the side strips 34a are symmetrical about the longitudinal centre line of the connector 30. Therefore the central strip 32 is offset from both of, and does not overlap with either of, the side strips 34a. Instead, the central strip 32 is opposite to, alternately, the cross strips 34a and the spaces between them. The cross strips 34b have a width b in the longitudinal direction along the interconnect 30, and the period of the spaces, i.e. the sum of the widths of one cross strip and one space, is a. The central conducting strip 32 therefore alternates, along its length, between capacitive regions, which are opposite the cross strips 34b of the ground layer 34, and non-capacitive regions which are opposite the spaces in the ground layer 34.

This arrangement is therefore an example of a modified micro-strip whose ground plane 34 is patterned with periodic openings or spaces, where no metal is present, at regularly spaced regions along its length. In this case the openings are arranged asymmetrically to balance any induced voltages. The larger separation (b−a) between the cross strip conductors 34b in the open regions is arranged to increase average inductance and decrease average capacitance, allowing impedance to be controlled by the ratio b/a, where a is the period of the ground plane pattern and b is the length of the regions where the two conductors, on opposite sides of the insulator strip 36, overlap. However, the capacitance is now mainly defined in the overlap regions, stabilising the impedance against variations in the surround.

Referring to FIG. 4 in an interconnect according to a further embodiment of the invention, top and bottom conductors 42, 44 are both periodically patterned into meander layouts to give the planar equivalent of twisted-pair. Specifically, the top conductor 42 is formed from a series of cross strips 42b and edge strips 42a. The cross strips are equally spaced along the length of the interconnect 40 and each of which extends across the full width of the insulating strip 46. Each cross strip 42b is connected to the one adjacent to it on one side by an edge strip 42a extending along one edge of the insulating strip 46, and to the one on the other side by an edge strip 42a extending along the other edge of the insulating strip 46. The resulting conducting layer 42 meanders in a square wave shape. The bottom conductor forming the ground layer is of the same shape, being made up of edge strips 44a and cross strips 44b. The cross strips 42b, 44b of the two layers are arranged to overlap, forming capacitor regions, and the top layer edge strip 42a between each pair of cross strips 42b is adjacent to the opposite edge of the insulating strip to the bottom layer edge strip 44a between the corresponding pair of edge strips 42a. Therefore each of the edge strips includes at least a region which does not overlap with an edge strip on the opposite side of the insulating strip. This arrangement allows serial balancing of induced voltages. Obviously, as with the arrangement of FIG. 3, the width of the cross strips and their spacing can be tuned to obtain the required impedance.

It will be appreciated that other patterns can be used giving similar effect. For example either of the two layers 42, 44 in the embodiment of FIG. 4 could be replace by a single strip extending along one edge of the insulating strip 46, or along the centre of the insulating strip like that of FIG. 3.

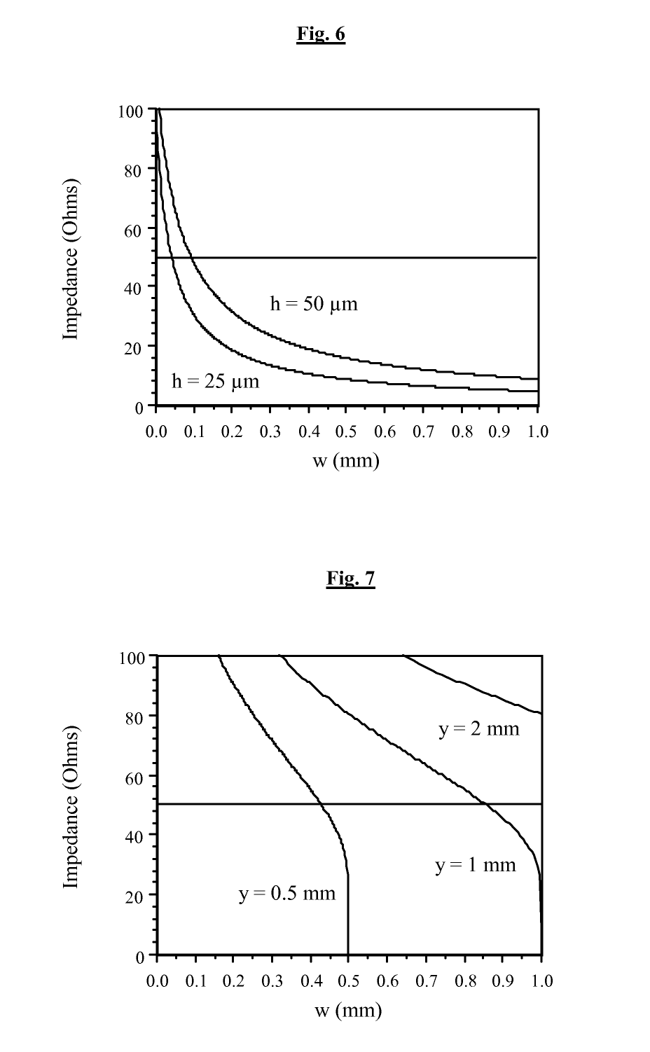

Either of the interconnects of FIGS. 3 and 4 can be connected directly to a thin-film resonant detector of the type described in GB 0910039.7, which can be formed on a part of the same substrate strip, as shown in FIG. 5 for the interconnect of FIG. 3. Here a two-turn rectangular inductor 50 is formed on the top surface of the insulating strip 36. A matching capacitor C. has one plate formed on the top surface of the insulating strip 36, connected on one side to the outer end of the inductor coil 50 and on the other side to the conducing strip 32 of the interconnect, and one plate formed on the back surface of the insulating strip 36 and connected to the ground layer 34. A tuning capacitor C_{T} is formed inside the coil 50, with one plate formed on the top surface of the conducting strip and connected to the inner end of the coil 50, and the other plate formed on the back surface of the insulating strip, and connected by a connecting strip to the back plate of the matching capacitor. The insulating substrate therefore provides the dielectric interlayer for the tow capacitors C_{T} C_{M}. The smaller capacitor C_{M} being located outside the spiral, and the larger capacitor C_{T} inside, avoids the need for an air-bridge or a via hole.

Referring to FIGS. 5a and 5b, the coil and interconnect system of FIG. 5 can be mounted on a catheter 58, being wrapped around its outer surface. The catheter 58 may be about 2 meters long, and in the form of a long cylindrical body with lumens, in this case two lumens 59, extending along it. The width of the insulating strip 36 is chosen to be slightly greater than half the circumference of the catheter. Referring to FIG. 5a, this means that the two side strips of the ground plane are located approximately on opposite sides of the catheter. Referring to FIG. 5b, the two turns of the inductor coil 50 are of such a width that one is slightly wider than half the circumference of the catheter and one is slightly narrower than half the circumference of the catheter. Therefore, one side of each turn extends along one side of the catheter and the other side extends along the other. The interconnect and coil system is enclosed in a heat-shrink tube 60 which secures it to the catheter and provides liquid-proof protection for it.

A line with a periodically patterned ground plane, such as those of FIGS. 3 and 4, is a bi-periodic structure, with different values of inductance and capacitance in alternating sections. Such a line is analogous to a diatomic lattice [Kittel C. “Introduction to solid state physics” Ch. 5 “Phonons and lattice vibrations” 3rd Ed., John Wiley and Sons, New York (1968)] and will generally have two propagating bands, as shown in the example dispersion diagram of FIG. 8. However, if the periodicity is sufficiently small and the component values in each section sufficiently different, the upper (optical) branch may be at very high frequencies. Since we are interested here only in low-frequency behaviour, we can ignore the bi-periodicity and simply assume that the impedance is dominated by an inductance L where the ground plane has been cut away and a capacitance C where it remains. Behaviour in the lower (acoustic) branch is then well approximated by the dispersion equation of an L-C ladder:

ω/ω₀=2 sin(ka/2) (5)

Here ω is the angular frequency, ω₀=1/√(LC) and k is now the propagation constant. Propagation can take place from DC to a maximum angular frequency ωₘ=2 ω₀. In this regime, the impedance is:

Z₀=Z₀′ exp(jka/2) (6)

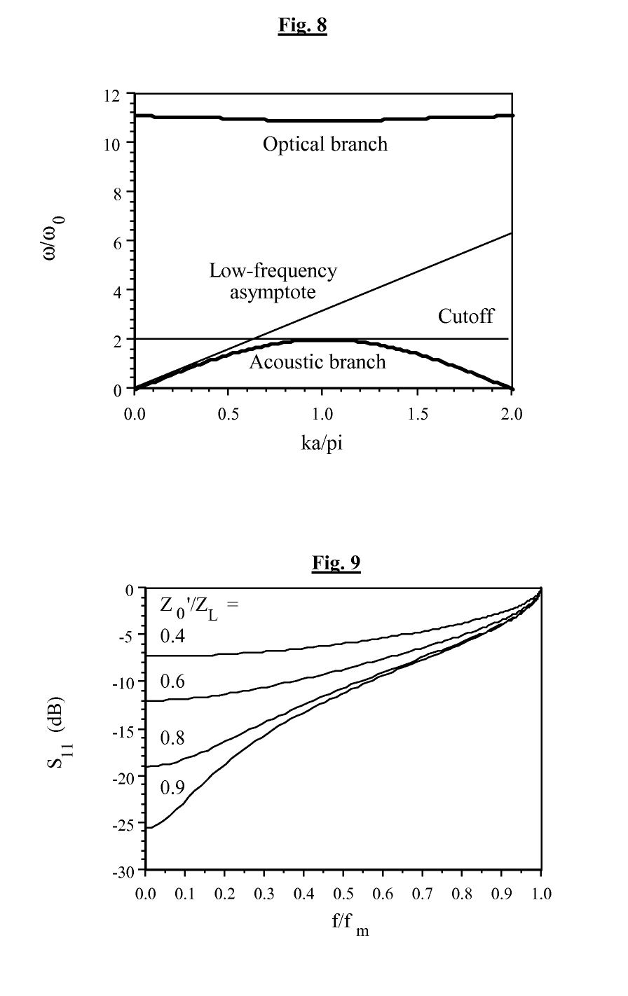

Here, Z₀′=√(L/C). The impedance is generally complex, but tends to the real value Z₀′ at low frequencies. The standard result S₁₁=(Z_{L}−Z₀)/(Z_{L}+Z₀) can be used to predict the scattering parameter S₁₁ at a junction with a terminating impedance Z_{L}. FIG. 9 shows the frequency variation of |S₁₁|² for different values of the ratio Z₀′/Z_{L}. The results show that good matching can be achieved at DC as Z₀′/Z_{L} tends to unity. However, the matching clearly degrades as the frequency rises, and all the power must be reflected at a cut-off frequency fₘ=ωₘ/2πn. For good low-frequency performance we should therefore have Z₀′/Z_{L} close to unity and fₘ much greater than the operating frequency f. Exactly how much greater depends on the range over which a given return is required.

Suitable design parameters may be estimated as follows. Using the geometric variables in FIG. 3, the DC impedance may be written as:

Z₀′=√{(a−b)Lₚ/bCₚ)} (7)

Here Lₚ and Cₚ are values per unit length. Low-frequency matching to impedance Z_{T} will be achieved if parameters can be chosen such that (a−b)Lₚ=Z_{L}²bCₚ. Since ωₘ can be written as:

ωₘ=2/√{(a−b)bLₚCₚ)} (8)

An impedance-matched line will therefore have ωₘ=2/(bZ_{L}Cₚ). To obtain reasonably constant impedance, operation might be restricted to frequencies below a given fraction of the cut-off frequency—say, f<fₘ/40. The maximum allowed length b of capacitance per section is then:

b<1/(20πfZ_{L}Cₚ) (9)

The overall period may then be found from the inductance Lₚ p.u.l. as:

a=b{1+Z_{L}²Cₚ/Lₚ} (10)

Estimation of C_{P} is relatively simple, since a parallel plate model may be used for wide strips on a thin substrate. For the previous parameters of w =1 mm, h=25 μm and εᵣ=3.5, C_{P}≈1.24 pF/mm. At 63.8 MHz (the operating frequency for ¹H MRI in a 1.5 T field) we then obtain fₘ=2.55 GHz and b=2 mm.

Estimation of L_{P} is harder, since the inner conductor is a strip and the outer conductors are sheets whose shapes and orientations will modify as the substrate is wrapped around a catheter. However, if we ignore the fact that the conductors lie on opposite sides of a thin sheet mounted on a curved support, and simply assume that the strip is mounted on a flat dielectric, the arrangement has a strong similarity to a CPW structure. We may therefore use Equations 3 and 4 to estimate the capacitance in the open areas, and then find the per-unit-length inductance as Lₚ=Z₀′²Cₚ. For y=2 mm, w=1 mm and εᵣ=3.5, we obtain Lₚ≈0.4 nH/mm. Assuming that Z_{L}=50Ω, the overall period can then be found as a=18 mm. These considerations suggest that centimetric periods and b/a ratios of around ⅛ will be suitable. In any case, re-arrangement of earlier results gives:

Cₚ=2/(bπfₘZ₀′),

Lₚ={b/(a−b)}CₚZ₀′² (11)

Actual values of Cₚ and Lₚ may therefore be extracted from experimental measurement of the DC impedance Z₀′ and the cut-off frequency fₘ.

Experimental Results

Periodically patterned interconnects with the arrangement of FIG. 3 were fabricated in continuous 2 metre lengths by the flexible PCB company Clarydon (Willenhall, West Midlands, UK) using reel-to-reel processing. Each line consisted of a 35 μm Cu thick strip conductor on a 25 μm thick Kapton® sheet with a 35 μm thick Cu ground. Experiments were also carried out using 50 μm Kapton®, but this resulted in stiffer interconnects that required thick heat-shrink for retention. A period close to the previous estimate was used (a=16 mm) so that each line contained 125 periods. A conductor strip width of w=1 mm was used, together with seven values of the ratio b/a (1, ½, ¼, ⅛, 1/16, 1/32 and 1/64). The first value corresponds to a uniform microstrip with no spaces in the ground layer, and the others to periodically patterned lines. Experiments were also carried out using 0.5 mm wide conductor strips, but these suffered from occasional line breaks. The ground plane width was 4 mm, and the defect width was y=2 mm. Lines were fabricated in arrays as shown in FIG. 10 and separated using a scalpel.

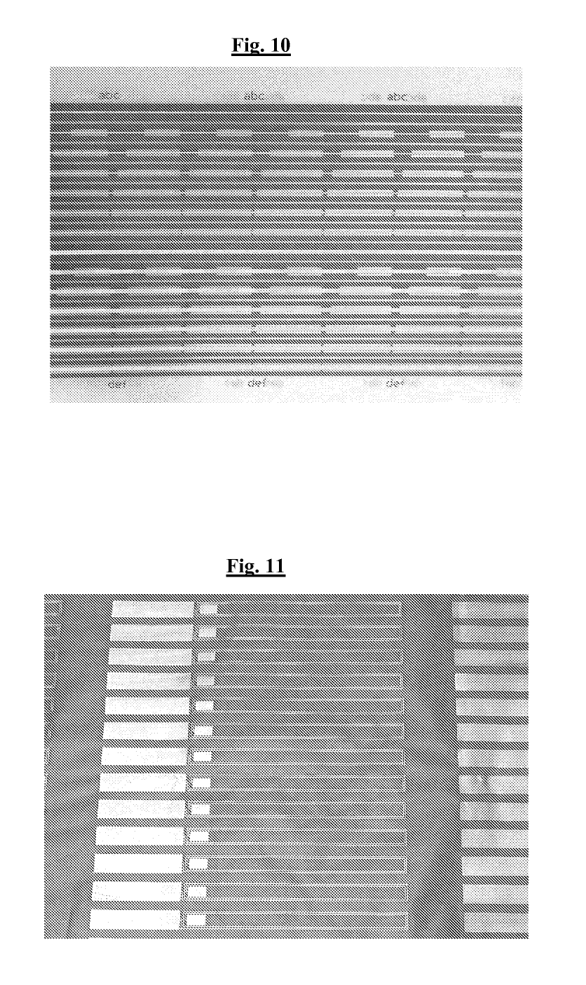

Resonant RF detectors with the layout of FIG. 5 were constructed on separate sheets, using similar materials and processing. The inductors consisted of two-turn rectangular spirals with a conductor width of 250 μm and separation of 250 μm, and a coil length of 60 mm and width of 4.2 mm. The coil width was chosen to place the long conductors, i.e. the sides of the coil, on the diameter of an 8 Fr catheter, as shown in FIG. 5b. Capacitor areas of the matching and tuning capacitors were chosen for operation at 63.8 MHz frequency, using small adjustments in area to achieve precise tuning and matching as described in GB 0910039.7. 120 similar resonators were formed on a panel measuring 300 mm×450 mm, as shown in FIG. 11 and again separated using a scalpel.

Connections to the flexible interconnect were made by forming a small slit from the end of the insulating strip 36, parallel to and just to one side of the strip conductor 32. The detector was inserted into this slit so that the part of the interconnect to one side of the slit lay over the detector, and part of the interconnect to the other side of the slit lay under the detector, and so that the ground plane just to one side of the slit, and the strip just to the other side of the slit, contacted the top and bottom plates of the matching capacitor, and solder was used to form a permanent joint.

Complete systems were then attached to catheters as shown in FIG. 1f. The large size of C_{M} (30 mm×6 mm) increased the film stiffness near the resonant detector. Improvements could be achieved reducing capacitor areas using a thinner substrate, and we have already demonstrated similar coils on 13 μm thick Kapton® with approximately half the capacitor area.

Electrical performance was assessed using an Agilent E5071B network analyser. Flexible inter-connects were first measured in isolation, before and after mounting on a catheter. Similar performance trends were obtained in each case, with any changes being attributable to a reduction in the inductance of patterned lines after being wrapped round a cylindrical former. Catheter-mounted lines were stable and could be flexed without significant variation in S-parameter measurements. The uniform microstrips had low impedance, as expected. Variants with periodically patterned ground planes offered significantly better matching at low frequency. Generally the matching improved as the ratio b/a decreased until an optimum was reached; if b/a was reduced further, the matching degraded. For h=25 μm and w=1 mm, the optimum value was b/a=⅛, in agreement with earlier estimates.

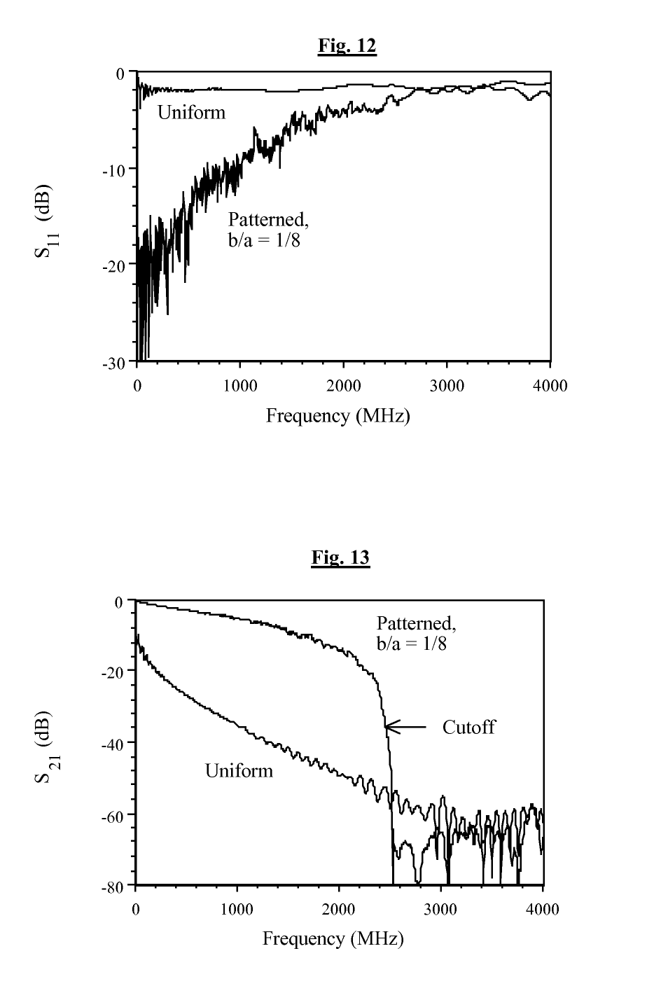

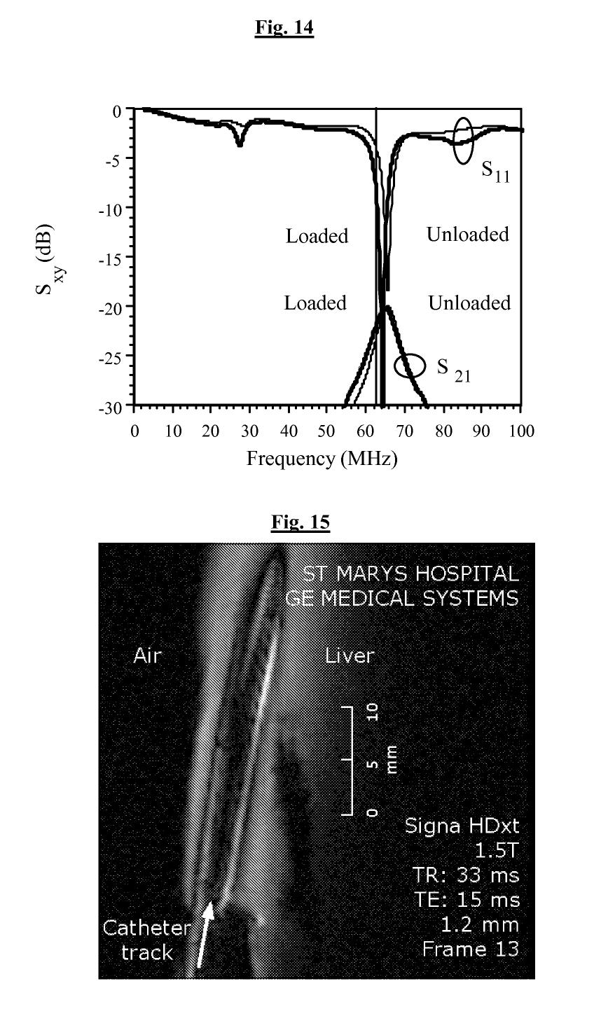

FIGS. 12 and 13 compare the frequency variation of S-parameters for uniform and patterned microstrips. Here the measurements are taken up to the GHz regime to allow determination of the cutoff frequency. For the uniform microstrip, the reflection is high and oscillations in S₁₁ and S₂₁ can be seen at low frequencies due to multiple reflections. At high frequencies, when losses are high enough that multiple reflections can be ignored, the return is S₁₁≈−1.9 dB. The characteristic impedance may then be estimated as 5.5 Ω. This value may be compared with an estimate of 4.5Ω obtained from FIG. 6.

For the periodically patterned interconnect, S₁₁ is much lower at low frequencies, with a peak value below −15 dB up to 500 MHz, rising almost to 0 dB at the cutoff frequency, at which point the transmission S₂₁ falls. The upper (optical) band was not identified, suggesting that this must lie at very high frequencies. The capacitance and inductance per unit length were estimated from the DC impedance (45 Ω) and the cut-off frequency (2.5 GHz) as 2.8 pF/mm and 0.8 nH/mm, respectively, in agreement with earlier estimates. Generally, performance of patterned microstrip was as expected; however, it also offered much lower propagation loss. In fact, the optimum line in FIG. 13 has low-frequency losses as low as 2.5 dB GHz⁻¹m⁻¹, making the losses negligible at 63.8 MHz.

Resonant RF detectors were measured both in isolation and after connection to a periodically patterned interconnect. Resonant frequency and impedance were first adjusted for operation at 63.8 MHz by slight variation of capacitor areas using conductive epoxy, and Q-factors were determined as ≈25 for an unloaded device. The overall response of complete thin-film detector systems was then measured. FIG. 14 shows the frequency variation of S₁₁ for the combination of a resonant detector and a periodically patterned interconnect. Here the detector has been tuned to a frequency approximately 2 MHz too high, so that the resonance decreases to 63.8 MHz when the detector is loaded by insertion into tissue such as a liver duct.

FIG. 14 also shows the frequency variation of S₂₁ obtained using a small inductive loop as a source and the catheter probe as a detector.

¹H magnetic resonance imaging was demonstrated using a 1.5 T GE HD Signa Excite scanner. The system body coil was used for transmission and the thin film detector system was connected to an auxiliary coil input for reception. The object was a butchered lamb's liver, and the microcoil was located in an accessible biliary duct. The microcoil was located at the magnet isocentre with the long conductors lying in the coronal plane, and imaging was demonstrated using a fast recovery fast spin echo (FRFSE) sequence. Imaging was carried out using a relaxation recovery time (TR) of 33 ms, an echo time (TE) of 15 ms and a flip angle of 10°. The images were acquired using a T₂-weighted FRFSE sequence in 28 slices of 1.2 mm thickness, a 100 mm field of view. FIG. 15 is a typical sagittal slice, which shows the track of the catheter inside the duct. These results are comparable to those obtained using a co-axial output cable, and demonstrate practical operation of the integrated thin film detector system.

It can therefore be seen that, using embodiments of the invention, a complete RF microcoil detector system can be designed for in-vivo internal magnetic resonance imaging, based on a thin film resonant detector and a thin-film microstrip, using periodic defects in the ground plane to achieve impedance matching at low frequency. Fabrication can be carried out using double-sided processing of a Cu-Kapton-Cu trilayer to yield a flexible strip designed for attachment to a catheter with heat-shrink tubing. Although two different substrates were used, one for the interconnect and one for the coil, in the examples describe above, the arrangement is suitable for integration on a common substrate, allowing complete detector systems to be formed by continuous patterning. These detectors may then be integrated onto catheter tools at low cost. There is scope for further miniaturisation using thinner substrates and narrower interconnects, with appropriate impedances being obtained by scaling, and for increasing high-frequency performance using periodic lines with smaller periods.

1-13. (canceled)

14. An interconnect for an RF detector coil, the interconnect comprising:
a substrate comprising a strip of insulating material and having a first side and a second side;
a conducting layer formed on the first side of the substrate; and
a ground layer formed on the second side of the substrate, wherein at least one of the layers has a periodic pattern so that the conducting layer comprises capacitive regions which are opposite regions of the ground layer, and non-capacitive regions, the capacitive regions and non-capacitive regions alternating along a length of the conducting layer.

15. The interconnect according to claim 1 wherein the second side of the substrate is only partly covered by the ground layer and has parts which are not covered by the ground layer, and in the non-capacitive regions the conducting layer is opposite the parts of the second side which are not covered by the ground layer.

16. The interconnect according to claim 1 wherein the substrate is flexible.

17. The interconnect according to claim 1 wherein the conducting layer is in the form of a straight strip.

18. The interconnect according to claim 1 wherein the ground layer is patterned so as to provide the capacitive and non-capacitive regions of the conducting layer.

19. The interconnect according to claim 18 wherein the ground layer comprises two spaced apart longitudinal strips extending along the substrate with cross strips extending across the substrate between the longitudinal strips.

20. An RF detector coil assembly comprising an interconnect according to claim 1 and a detector coil, the coil having two ends, one of which ends is connected to the conducting layer and another of which ends is connected to the ground layer.

21. The assembly according to claim 20 wherein the coil is formed on a flexible substrate.

22. The assembly according to claim 20 wherein the coil is formed on the strip of insulating material.

23. A catheter assembly comprising a catheter having a cylindrical body having an outer surface, and an interconnect according to claim 1 wrapped around the outer surface.

24. The catheter assembly according to claim 23 further comprising a detector coil connected to the interconnect.

25. The catheter assembly comprising a catheter body having an outer surface and a detector coil assembly according to claim 20 wrapped around said outer surface.

26. The catheter assembly according to claim 20 further comprising a protective sleeve enclosing the interconnect and the catheter body.
